(19) United States
(12) Patent Application Publication
(10) Pub. No.: US 2006/0217593 A1
(43) Pub. Date: Sep. 28, 2006

(54) Device, system and method of panoramic multiple field of view imaging

(76) Inventors: Zvika Gilad, Haifa (IL); Gavriel J. Iddan, Haifa (IL)

Correspondence Address: PEARL COHEN ZEDEK, LLP, 1500 BROADWAY 12TH FLOOR, NEW YORK, NY, 10036, US

(21) Appl. No.: 11/385,901

(22) Filed: Mar. 22, 2006

Related U.S. Application Data
(60) Provisional application No. 60/664,591, filed on Mar. 24, 2005.

(51) Int. Cl. A61B 1/06

(52) U.S. Cl. 600/160

(57) ABSTRACT

Device, system and method of panoramic multiple field of view imaging. For example, an in-vivo imaging device includes an imager able to acquire an in-vivo image including at least a first image-portion and a second image-portion, the first image-portion corresponding to a first field of view of the in-vivo imaging device, and the second image-portion corresponding to a second field of view of the in-vivo imaging device

### PRIOR APPLICATION DATA

This application claims benefit and priority from U.S. Provisional Patent Application No. 60/664,591, filed on Mar. 24, 2005, entitled “Device, System and Method of Panoramic Multiple Field of View Imaging”, which is hereby incorporated by reference in its entirety.

### FIELD OF THE INVENTION

The present invention relates to the field of in-vivo sensing, for example, in-vivo imaging.

### BACKGROUND OF THE INVENTION

Some in-vivo sensing systems may include an in-vivo imaging device able to acquire and transmit images of, for example, the GI tract while the in-vivo imaging device passes through the GI lumen.

Other devices, systems and methods for in-vivo sensing of passages or cavities within a body, and for sensing and gathering information (e.g., image information, pH information, temperature information, electrical impedance information, pressure information, etc.), are known in the art.

Some in-vivo imaging devices may have a limited field-of-view.

### SUMMARY OF THE INVENTION

Some embodiments of the invention may include, for example, devices, systems, and methods for obtaining a panoramic or circular (e.g., substantially 360 degrees, or other ranges) field-of-view.

Some embodiments of the invention may include, for example, an in-vivo imaging device having a reflective element, which may be curved or may have a non-flat shape. In some embodiments, the reflective element may reflect light rays from an imaged object or lumen onto an imager, where such light rays may be, before being reflected, substantially parallel to a plane of such imager.

In some embodiments, for example, the imager may capture panoramic, substantially panoramic, or partially panoramic images of an in-vivo area, object or lumen. In some embodiments, for example, an acquired image may approximate a ring-shaped slice of a body lumen.

In some embodiments, for example, the in-vivo imaging device may include illumination units arranged around an inside perimeter of the in-vivo imaging device. In some embodiments, for example, illumination units may be situated on an outward-facing ring, such that the illumination units are directed outwards from the in-vivo imaging device. In other embodiments, light may be generated by an illumination source which may be external to the in-vivo imaging device.

In some embodiments, for example, the in-vivo imaging device may include a concave, tapered, narrowed shaped portion, such that the in-vivo imaging device may have a “peanut” like shape. In some embodiments, for example, the narrowed or concave portion may include a transparent ring around an outer shell of the in-vivo imaging device.

Some embodiments of the invention include, for example, an in-vivo imaging device having a reflective surface that may be situated at an angle, e.g., approximately 45 degrees angle relative to the plane of an imager of the in-vivo imaging device. In some embodiments, the reflective surface may reflect light rays onto an imager, where such light rays before reflection were substantially parallel to the plane of the imager.

In some embodiments, for example, the reflective surface may be rotated by, e.g., a motor, and may allow acquisition of images having a panoramic, substantially panoramic, or partially panoramic field-of-view of an object or body lumen. In some embodiments, for example, illumination of a body lumen or object may be substantially synchronized with such rotation, and may provide, for example, substantially homogenous illumination of an in-vivo area or body lumen. In some embodiments, the rotation may be at a substantially constant rate or at a variable rate.

In some embodiments, for example, the field-of-view imaged by the in-vivo imaging device may include an area substantially perpendicular to the in-vivo imaging device, an area in front of the in-vivo imaging device, and/or an area behind the in-vivo imaging device.

In some embodiments, a panoramic image may be flattened or otherwise converted into a substantially rectangular image, and may be displayed, e.g., on an external display system or monitor.

Some embodiments may include, for example, an in-vivo imaging device able to view and/or capture images of body areas transverse and/or substantially transverse to the general direction of movement of the in-vivo imaging device.

In some embodiments, for example, the in-vivo imaging device may include a reflective element having an aperture, allowing an imager to acquire an image having multiple portions. The aperture may allow light rays to pass from a frontal field of view (e.g., having a body lumen, an object, a sensor, or the like) onto the imager, e.g., a field of view which may be along the larger axis of the in-vivo imaging device or “in front of” the imager. For example, in one embodiment, a first portion of the image may include a panoramic image of light reflected from the reflective element; a second portion of the image may include an image of a sensor having a visual indication related to its sensing; and a third portion of the image may include an image of a frontal field-of-view of the imager.

Some embodiments of the invention further include a method and a system for using such in-vivo imaging devices.

In some embodiments, the in-vivo imaging device may include, for example, an autonomous in-vivo device and/or a swallowable capsule.

Embodiments of the invention may provide various other benefits or advantages.

### BRIEF DESCRIPTION OF THE DRAWINGS

The subject matter regarded as the invention is particularly pointed out and distinctly claimed in the concluding portion of the specification. The invention, however, both as to organization and method of operation, together with containers, features, and advantages thereof, may best be understood by reference to the following detailed description when read with the accompanying drawings in which:

FIG. 1 is a schematic illustration of an in-vivo imaging system in accordance with some embodiments of the invention;

FIG. 2 is a schematic illustration of an in-vivo imaging device having a reflective element in accordance with some embodiments of the invention;

FIGS. 3A-3E are schematic illustrations helpful to understanding some aspects of the operation of an in-vivo imaging device in accordance with some embodiments of the invention;

FIG. 4A is a schematic illustration of an in-vivo imaging device having a narrowed section in accordance with some embodiments of the invention;

FIG. 4B is a schematic illustration of a series of Light Emitting Diodes that are situated on a ring that is slanted outward in accordance with some embodiments of the invention;

FIG. 5 is a flow chart diagram of a method of capturing an image using a curved reflective element in accordance with some embodiments of the invention;

FIG. 6 is a schematic illustration of an in-vivo imaging device including a rotating mirror in accordance with some embodiments of the invention;

FIG. 7 is a flow chart of a method of reflecting onto an imager light rays that are substantially parallel to the imager, in accordance with some embodiments of the invention;

FIG. 8 is a depiction of a panoramic in-vivo imaging device in accordance with an some embodiments of the invention;

FIG. 9 is a schematic illustration of an in-vivo imaging device able to acquire images from one or more sources or from one or more fields-of-view, in accordance with some embodiments of the invention; and

FIG. 10 is a schematic illustration of an exemplary image which may be captured by the in-vivo imaging device of FIG. 9.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, components and circuits have not been described in detail so as not to obscure the present invention.

Although a portion of the discussion may relate to in-vivo imaging devices, systems, and methods, the present invention is not limited in this regard, and some embodiments of the present invention may be used in conjunction with various other in-vivo sensing devices, systems, and methods. For example, some embodiments of the invention may be used, for example, in conjunction with in-vivo sensing of pH, in-vivo sensing of temperature, in-vivo sensing of pressure, in-vivo sensing of electrical impedance, in-vivo detection of a substance or a material, in-vivo detection of a medical condition or a pathology, in-vivo acquisition or analysis of data, and/or various other in-vivo sensing devices, systems, and methods.

Some embodiments of the present invention are directed to a typically one time use or partially single use detection and/or analysis device. Some embodiments are directed to a typically swallowable in-vivo device that may passively or actively progress through a body lime, e.g., the gastro-intestinal (GI) tract, for example, pushed along by natural peristalsis. Some embodiments are directed to in-vivo sensing devices that may be passed through other body lumens, for example, through blood vessels, the reproductive tract, or the like. The in-vivo device may be, for example, a sensing device, an imaging device, a diagnostic device, a detection device, an analysis device, a therapeutic device, or a combination thereof. In some embodiments, the in-vivo device may include an image sensor or an imager. Other sensors may be included, for example, a pH sensor, a temperature sensor, a pressure sensor, sensors of other in-vivo parameters, sensors of various in-vivo substances or compounds, or the like

Devices, systems and methods according to some embodiments of the present invention, including for example in-vivo sensing devices, receiving systems and/or display systems, may be similar to embodiments described in U.S. Pat. No. 5,604,531 to Iddan et al., entitled “In-vivo Video Camera System”, and/or in U.S. Pat. No. 7,009,634 to Iddan et al., entitled “Device for In-Vivo Imaging”, and/or in U.S. patent application Ser. No. 10/046,541, entitled “System and Method for Wide Field Imaging of Body Lumens”, filed on Jan. 16, 2002, published on Aug. 15, 2002 as United States Patent Application Publication Number 2002/0109774, and/or in U.S. patent application Ser. No. 10/046,540, entitled “System and Method for Determining In-vivo Body Lumen Conditions”, filed on Jan. 16, 2002, published on Aug. 15, 2002 as United States Patent Application Publication Number 2002/0111544, all of which are hereby incorporated by reference in their entirety. Devices and systems as described herein may have other configurations and/or sets of components. For example, an external receiver/recorder unit, a processor and a monitor, e.g., in a workstation, such as those described in the above publications, may be suitable for use with some embodiments of the present invention. Devices and systems as described herein may have other configurations and/or other sets of components. For example, the present invention may be practiced using an endoscope, needle, stent, catheter, etc. Some in-vivo devices may be capsule shaped, or may have other shapes, for example, a peanut shape or tubular, spherical, conical, or other suitable shapes.

Some embodiments of the present invention may include, for example, a typically swallowable in-vivo device. In other embodiments, an in-vivo device need not be swallowable and/or autonomous, and may have other shapes or configurations. Some embodiments may be used in various body lumens, for example, the GI tract, blood vessels, the urinary tract, the reproductive tract, or the like. In some embodiments, the in-vivo device may optionally include a sensor, an imager, and/or other suitable components.

Embodiments of the in-vivo device are typically autonomous and are typically self-contained. For example, the in-vivo device may be or may include a capsule or other unit where all the components are substantially contained within a container, housing or shell, and where the in-vivo device does not require any wires or cables to, for example, receive power or transmit information. The in-vivo device may communicate with an external receiving and display system to provide display of data, control, or other functions. For example, power may be provided by an internal battery or an internal power source, or using a wired or wireless power-receiving system. Other embodiments may have other configurations and capabilities. For example, components may be distributed over multiple sites or units; and control information or other information may be received from an external source.

Devices, systems and methods in accordance with some embodiments of the invention may be used, for example, in conjunction with a device which may be inserted into a human body or swallowed by a person. However, embodiments of the invention are not limited in this regard, and may be used, for example, in conjunction with a device which may be inserted into, or swallowed by, a non-human body or an animal body.

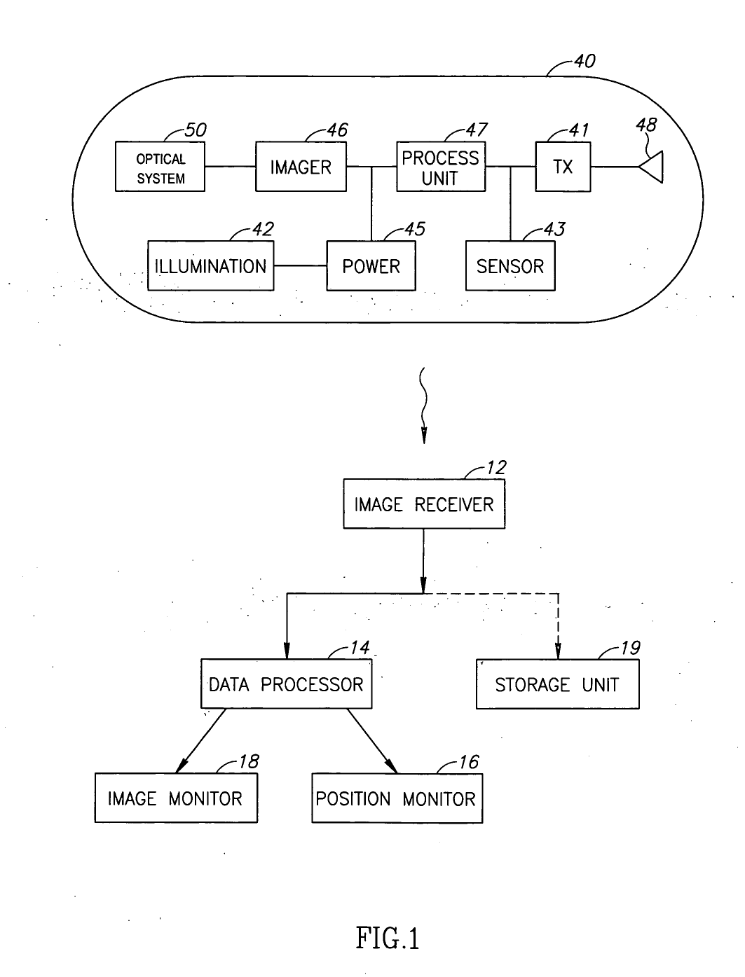

Reference is made to FIG. 1, which shows a schematic diagram of an embodiment of an in-vivo imaging system. In one embodiment, the system may include a device 40 having an imager 46, an illumination source 42, and a transmitter 41 with an antenna 48. In some embodiments, device 40 may be implemented using a swallowable capsule, but other sorts of devices or suitable implementations may be used. Outside the patient's body may be an image receiver 12 (typically including an antenna or an antenna array), a storage unit 19, a data processor 14, an image monitor 18, and a position monitor 16. While FIG. 1 shows separate monitors, in some embodiments, both an image and its position may be presented using a single monitor. Other systems and methods of storing and/or displaying collected image data may be used.

Transmitter 41 may typically operate using radio waves, but in some embodiments, such as those where the device 40 is or is included within an endoscope, transmitter 41 may transmit via, for example, wire.

Device 40 typically may be or include an autonomous swallowable imaging device such as for example a capsule, but may have other shapes, and need not be swallowable or autonomous. In one embodiment, device 40 may include an in-vivo video camera which may capture and transmit images of the GI tract while the device passes through the GI lumen. Other lumens may be imaged.

Imager 46 in device 40 may be connected to transmitter 41 also located in device 40. Transmitter 41 may transmit images to image receiver 12, which may send the data to data processor 14 and/or to storage unit 19. Transmitter 41 may also include control capability, although control capability may be included in a separate component. Transmitter 41 may include any suitable transmitter able to transmit images and/or other data (e.g., control data) to a receiving device. For example, transmitter 41 may include an ultra low power RF transmitter with high bandwidth input, possibly provided in Chip Scale Package (CSP). Transmitter 4 may transmit via antenna 48.

A system according to some embodiments of the invention includes an in-vivo sensing device transmitting information (e.g., images or other data) to a data receiver and/or recorder possibly close to or worn on a subject. A data receiver and/or recorder may of course take other suitable configurations. The data receiver and/or recorder may transfer the information received from a transmitter to a larger computing device, such as a workstation or personal computer, where the data may be further analyzed, stored, and/or displayed to a user. In other embodiments, each of the various components need not be required; for example, an internal device may transmit or otherwise transfer (e.g., by wire) information directly to a viewing or processing system.

In some embodiments, transmitter 41 may include, for example, a transmitter-receiver or a transceiver, to allow transmitter 41 to receive a transmission. Additionally or alternatively, a separate or integrated receiver (not shown) or transceiver (not shown) may be used within device 40, instead of transmitter 41 or in addition to it, to allow device 40 to receive a transmission. In one embodiment, device 40 and/or transmitter 41 may, for example, receive a transmission and/or data and/or signal which may include commands to device 40. Such commands may include, for example, a command to turn on or turn off device 40 or any of its components, a command instructing device 40 to release a material, e.g., a drug, to its environment, a command instructing device 40 to collect and/or accumulate a material from its environment, a command to perform or to avoid performing an operation which device 40 and/or any of its components are able to perform, or any other suitable command. In some embodiments, the commands may be transmitted to device 40, for example, using a pre-defined channel and/or control channel. In one embodiment, the control channel may be separate from the data channel used to send data from transmitter 41 to receiver 12. In some embodiments, the commands may be sent to device 40 and/or to transmitter 41 using receiver 12, for example, implemented using a transmitter-receiver and/or transceiver, or using a separate and/or integrated transmitter or transceiver in the imaging system.

Power source 45 may include, for example, one or more batteries or power cells. For example, power source 45 may include silver oxide batteries, lithium batteries, other suitable electrochemical cells having a high energy density, or the like. Other suitable power sources may be used. For example, in some embodiments (e.g., where device 40 is, or is included in, an endoscope) power source 45 may receive power or energy from an external power source (e.g., an electromagnetic field generator), which may be external to device 40 and/or external to the body, and may be used to transmit power or energy to in-vivo device 40.

In some embodiments, power source 45 may be internal to device 40, and/or may not require coupling to an external power source, e.g., to receive power. Power source 45 may provide power to one or more components of device 40, for example, continuously, substantially continuously, or in a non-discrete manner or timing, or in a periodic manner, an intermittent manner, or an otherwise non-continuous manner. In some embodiments, power source 45 may provide power to one or more components of device 40, for example, not necessarily upon-demand, or not necessarily upon a triggering event or an external activation or external excitement.

Data processor 14 may analyze the data and may be in communication with storage unit 19, transferring data such as frame data to and from storage unit 19. Data processor 14 may also provide the analyzed data to image monitor 18 and/or position monitor 16, where a user may view the data. In one embodiment, for example, image monitor 18 may present an image of the GI lumen, and position monitor 16 may present the position in the GI tract at which the image was taken. In one embodiment, data processor 14 may be configured for real time processing and/or for post processing to be performed and/or viewed at a later time. Other monitoring and receiving systems may be used in accordance with embodiments of the invention. Two monitors need not be used.

In some embodiments, in addition to revealing pathological conditions of the GI tract, the system may provide information about the location of these pathologies. Suitable tracking devices and methods are described in embodiments in the above mentioned U.S. Pat. No. 5,604,531 and/or U.S. Patent Application Publication No. US-2002-0173718-A1, filed May 20, 2002, titled “Array System and Method for Locating an In-Vivo Signal Source”, assigned to the assignee of the present invention, and fully incorporated herein by reference.

It is noted that in embodiments of the invention, other location and/or orientation detection methods may be used. In one embodiment, the orientation information may include three Euler angles or quaternion parameters; other orientation information may be used. In one embodiment, location and/or orientation information may be determined by, for example, including two or more transmitting antennas in device 40, each with a different wavelength, and/or by detecting the location and/or orientation using a magnetic method. In some embodiments, methods such as those using ultrasound transceivers or monitors that include, for example, three magnetic coils that receive and transmit positional signals relative to an external constant magnetic field may be used. For example, device 40 may include an optional location device such as tracking and/or movement sensor 43 to indicate to an external receiver a location of the device 40.

Optionally, device 40 may include a processing unit 47 that processes signals generated by imager 46. Processing unit 47 need not be a separate component; for example, processing unit 47 may be integral to imager 46 or transmitter 41, and may not be needed.

In some embodiments, device 40 may include one or more illumination sources 42, for example one or more Light Emitting Diodes (LEDs), “white LEDs”, monochromatic LEDs, Organic LEDs (O-LEDs), thin-film LEDs, single-color LED(s), multi-color LED(s), LED(s) emitting viewable light, LED(s) emitting non-viewable light, LED(s) emitting Infra Red (IR) light or Ultra Violet (UV) light, LED(s) emitting a light at a certain spectral range, a laser source, a laser beam(s) source, an emissive electroluminescent layer or component, Organic Electro-Luminescence (OEL) layer or component, or other suitable light sources

In some embodiments, an optional optical system 50, including, for example, one or more optical elements, such as one or more lenses or composite lens assemblies, one or more suitable optical filters (not shown), or any other suitable optical elements (not shown), may aid in focusing reflected light onto the imager 46 and performing other light processing. According to one embodiment optical system 50 includes a reflecting surface, such as a conical mirror.

Typically, device 40 transmits image information in discrete portions. Each portion typically corresponds to an image or frame. Other transmission methods are possible. For example, device 40 may capture an image once every half second, and, after capturing such an image, transmit the image to receiver 12. Other constant and/or variable capture rates and/or transmission rates may be used.

Typically, the image data recorded and transmitted may include digital color image data; in alternate embodiments, other image formats (e.g., black and white image data) may be used. In some embodiments, each frame of image data may include 256 rows, each row may include 256 pixels, and each pixel may include data for color and brightness according to known methods. According to other embodiments a 320×320 pixel imager may be used. Pixel size may be, for example, between 5 to 6 microns; other suitable sizes may be used. According to some embodiments, pixels may be each fitted with a micro lens. For example, a Bayer color filter may be applied. Other suitable data formats may be used, and other suitable numbers or types of rows, columns, arrays, pixels, sub-pixels, boxes, super-pixels and/or colors may be used.

In embodiments of the invention, device 40 and/or imager 46 may have a broad field-of-view. In some embodiments, device 40 and/or imager 46 may view and/or capture images of body areas transverse and/or substantially transverse to the general direction of movement of device 40. For example portions of body lumens directly adjacent to device 40, as opposed to in front of or behind the front and back (respectively) of device 40, may be imaged. Portions of body lumens between a forward and rear end of the device may be imaged. Furthermore, in some embodiments, device 40 and/or imager 46 may view and/or capture panoramic images with a broad field-of-view, e.g., up to 360 degrees, and/or with a substantially circular or radial field-of-view.

In some embodiments, device 40 may be configured to have a forward-looking field-of-view and/or a transverse field-of-view, for example, to produce a combined field-of-view having broad coverage both in line with device 40 and transverse thereto. In some embodiments, a transverse field-of-view may include in-vivo areas that are lying in planes that are perpendicular or substantially perpendicular to a plane of imager 46.

Embodiments of the invention may achieve a broad field-of-view, as detailed herein. Some embodiments may use a reflective element, for example, a curved or other suitably shaped mirror, to capture a panoramic image. A mirror or reflective element need not be curved or shaped. Some embodiments may use a rotating mirror or reflective element to capture a panoramic image. A rotating mirror or reflective element need not be curved or shaped. In some embodiments, a plurality of imagers may be used to capture a broad field-of-view, for example, by placing multiple imagers such that they face different and/or overlapping directions. In some embodiments, a rotating imager may be used to capture a panoramic image. It is noted that while some exemplary embodiments are explained in detail herein, the invention is not limited in this regard, and other embodiments and/or implementations of a broad field-of-view imaging device are also within the scope of the invention.

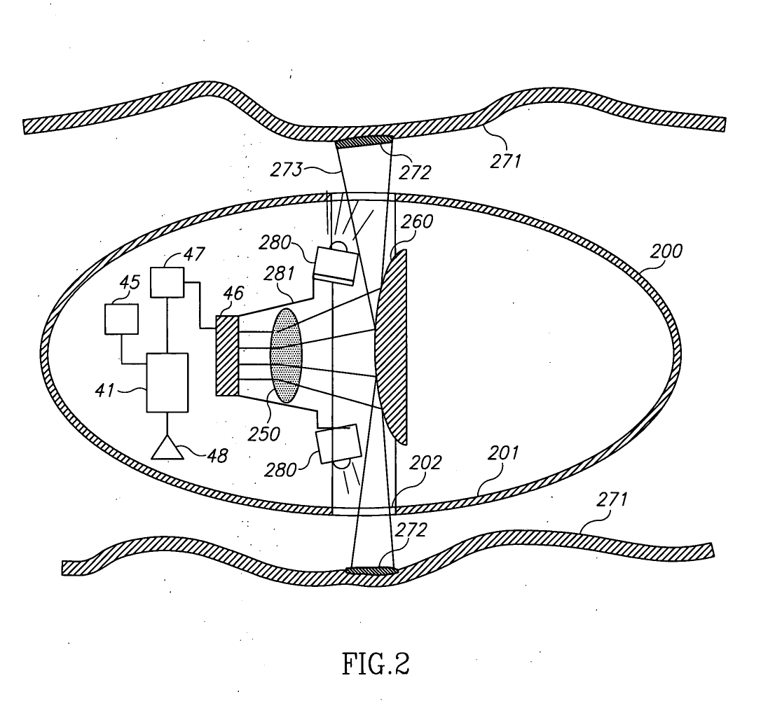

FIG. 2 is a schematic illustration of an in-vivo imaging device 200 in accordance with embodiments of the invention. Device 200 may be an implementation or variation of device 40, and may be used, for example, in conjunction with the system of FIG. 1 or certain components of FIG. 1. For example, device 200 may be used in conjunction with receiver 12 and/or data processor 14. In one embodiment of the invention, device 200 may include a device 200, e.g., a capsule or other suitable device, imager 46, a processing unit 47, a transmitter 41, an antenna 48, a power source 45, a lens assembly 250, a reflective element 260, an illumination source (or plurality of sources) 280, and a holder 281. The processing capability of processing unit 47 may be combined with other units, such as transmitter 41 or a separate controller.

In one embodiment of the invention, device 200 may be a swallowable capsule. Device 200 may be partially or entirely transparent. For example, device 200 may include areas, such as a transparent ring 202, which are transparent and which allow components inside device 200 to have an un-obstructed field-of-view of the environment external to device 200. According to one embodiment transparent ring 202 may be configured such that a 360 degree field of view is enabled. Other shaped transparent areas may be used; other sizes of a field of view may be used.

Imager 46 may include an electronic imager for capturing images. For example, imager 46 may include a Complimentary Metal Oxide Semiconductor (CMOS) electronic imager including a plurality of elements. In embodiments of the invention, imager 46 may include other suitable types of optical sensors and/or devices able to capture images, such as a Charge-Coupled Device (CCD), a light-sensitive integrated circuit, a digital still camera, a digital video camera, or the like. It is noted that a CMOS imager is typically an ultra low power imager and may be provided in Chip Scale Packaging (CSP). Other types of CMOS imagers may be used.

Processing unit 47 may include any suitable processing chip or circuit able to process signals generated by imager 46. For example, processing unit 47 may include a Central Processing Unit (CPU), a Digital Signal Processor (DSP), a microprocessor, a controller, a chip, a microchip, a controller, circuitry, an Integrated Circuit (IC), an Application-Specific Integrated Circuit (ASIC), or any other suitable multi-purpose or specific processor, controller, circuitry or circuit It is noted that processing unit 47 and imager 46 may be implemented as separate components or as integrated components; for example, processing unit 47 may be integral to imager 46. Further, processing may be integral to imager 46 and/or to transmitter 41.

In some embodiments, imager 46 may acquire in-vivo images, for example, continuously, substantially continuously, or in a non-discrete manner, for example, not necessarily upon-demand, or not necessarily upon a triggering event or an external activation or external excitement; or in a periodic manner, an intermittent manner, or an otherwise non-continuous manner.

In some embodiments, transmitter 41 may transmit image data continuously, or substantially continuously, for example, not necessarily upon-demand, or not necessarily upon a triggering event or an external activation or external excitement; or in a periodic manner, an intermittent manner, or an otherwise non-continuous manner.

Lens assembly 250 may include, for example, one or more lenses or optical systems which may allow imager 46 to focus on an image reflected by reflective element 260. Additionally or alternatively, lens assembly 250 may include a combination of lenses able to zoom in and/or zoom out on an image or magnify one or more parts of an image reflected by reflective element 260. Lens assembly 250 may include one or more optical elements, for example, one or more lenses and/or optical filters, to allow or to aid focusing reflected light onto imager 46 and/or performing other light processing operations.

Reflective element 260 may include, for example, a curved mirror. In some embodiments, reflective element 260 may include, for example, a metallic element, a reflective plastic element, a reflective coated plastic element, or a glass element. Reflective element 260 may be shaped and/or contoured such that it allows light reflected from a slice 272 of a body lumen 271 to be reflected by reflective element 260, through lens assembly 250, onto imager 46. For example, reflective element 260 may be oval, spherical, radial, circular, ellipse-shaped, faceted, conical, etc. It is noted that in some embodiments, reflective element 260 may have a shape, size and/or dimensions to allow a desired reflection of light and/or to allow a desired range and/or field-of-view. In one embodiment, reflective element 260 may be manufactured using suitable optical design software and/or ray-tracing software, for example, using “ZEMAX Optical Design Program” software. Other suitable shapes may be used.

Illumination source 280 may include one or more illumination sources or light sources to illuminate body lumen 271 and/or a slice 272 of body lumen 271. In one embodiment, illumination source 280 may include one or more Light-Emitting Diodes (LEDs), for example, one or more white LEDs. Such LEDs may be placed, aligned and/or positioned to allow a desired illumination of body lumen 271, for example, using a ring-shaped arrangement of LEDs able to illuminate body lumen 271 through transparent ring 202, that may for example be arranged around an inside perimeter of device 40. Other arrangements of illumination sources may be used in accordance with embodiments of the invention.

In some embodiments, an optional optical system may be used in conjunction with illumination source 280, for example, to create a desired illumination, for example, homogenous illumination, of an imaged body lumen. In one embodiment, the optical system may include, for example, one or more mirrors and/or curved mirrors and/or lenses and/or reflective elements, shaped and/or positioned and/or aligned to create a desired, e.g., homogenous, illumination. For example, in one embodiment, the optical system may include a curved mirror, similar to reflective element 260. According to further embodiments an optical system may include filters.

Holder 281 may include a suitable structure to hold illumination sources 280. In some embodiments, holder 281 may be formed and/or shaped such that it may reduce glare. In some embodiments, holder 281 may be formed and/or shaped such that it may block stray light from reaching and/or flooding imager 46.

In one embodiment, as device 200 traverses body lumen 271, device 200 may capture images of a slice of body lumen 271, such as slice 272. Illumination source 280 may illuminate slice 272 of body lumen 271. The light from illuminated slice 272 may be reflected using reflective element 260, focused and/or transferred using lens assembly 250, and received by imager 46 which may thereby capture an image of slice 272. Before they are reflected by reflective element 260, the light rays 273 reflected back from an illuminated object or illuminated slice 272 in an in vivo area, may be parallel or substantially parallel to the plane of imager 46 or an image sensor of device 200 upon which the light detection sensors are located. In some embodiments the angle at which light rays 273 may strike reflective element 260 may depend on the size of transparent ring 202. Other factors such as for example the placement of illumination source 280 and the distance of a wall of body lumen 271 from device 200 may also influence the angle at which light rays 273 are reflected onto reflective element 260. In some embodiments, the curvature of reflective element 260 may be fashioned so that light rays 273 striking reflective element 260 at various angles are reflected towards imager 46. Such curvature may affect the range of angles of light rays 273 that may be reflected by reflective element 260 onto imager 46. In some embodiments the in-vivo area of which images may be captured may be substantially perpendicular to the plane of an image sensor.

In one embodiment, since device 200 may include transparent areas and/or portions, such as transparent ring 202, the captured image may include a reflected image of a ring-shaped slice 272 of body lumen 271. It is noted that lens assembly 250 may be configured, placed and/or aligned to filter and focus light from body lumen 271, such that only or substantially only light from a desired portion of body lumen 271, for example, a ring-shaped slice 272, falls on imager 46. Using device 200 may allow, for example, capturing a panoramic image of slice 272 of body lumen 271. Such panoramic image may include a substantially complete 360 degrees image of slice 272. Alternatively, if desired, such image may include a non-complete image of slice 272, for example, a 270 degrees image, a 210 degrees image, a 180 degrees image, or any other number of degrees between 0 and 360.

In one embodiment, the panoramic image of slice 272 may be ring-shaped. Such an image may be converted into a rectangular image of slice 272 or into other shapes. In one embodiment, the conversion may be performed, for example, by processing unit 47 before transmitting the image. Additionally or alternatively, the conversion may be performed by an external processor such as data processor 14 after receiving the transmitted image. The conversion may be performed, for example, using methods as known in the art to “flatten” a ring-shaped image into a rectangular image. The conversion may include other suitable operations for image manipulation and/or image enhancement, performed before and/or after transmission of the image by transmitter 41 to receiver 12. The conversion may be applied to one image, or to a group or a batch of sequential or non-sequential images.

Additionally or alternatively, images of slices of body lumen 271, such as slice 272, may be placed, aligned and/or combined together, for example, side by side, to create a combined image or several combined images from a plurality of images of slices 272. The combination of images of slices 272 may be performed, for example, by processing unit 47 and/or data processor 14. Additionally or alternatively, the combination of images of slices 272 may be performed before and/or after transmission of the image by transmitter 41 to receiver 12.

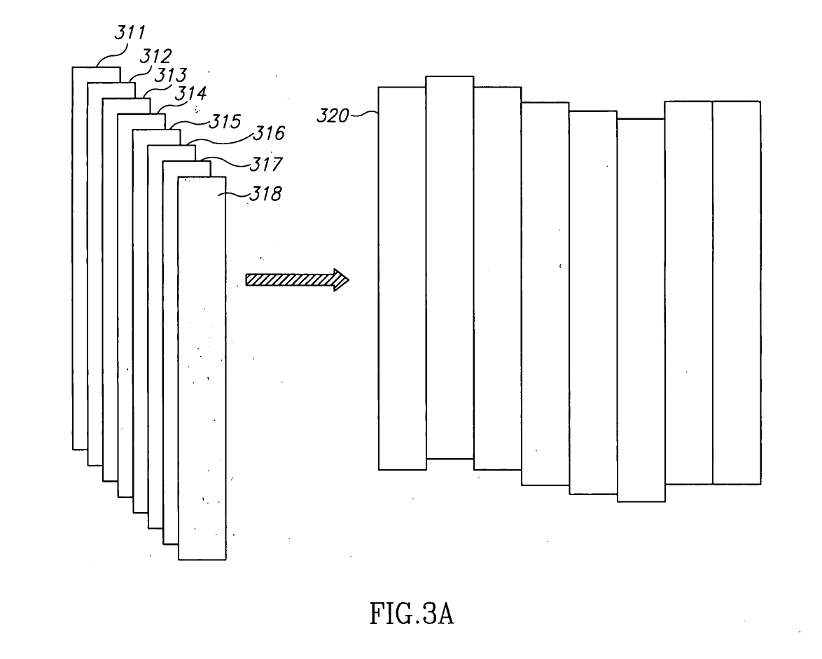

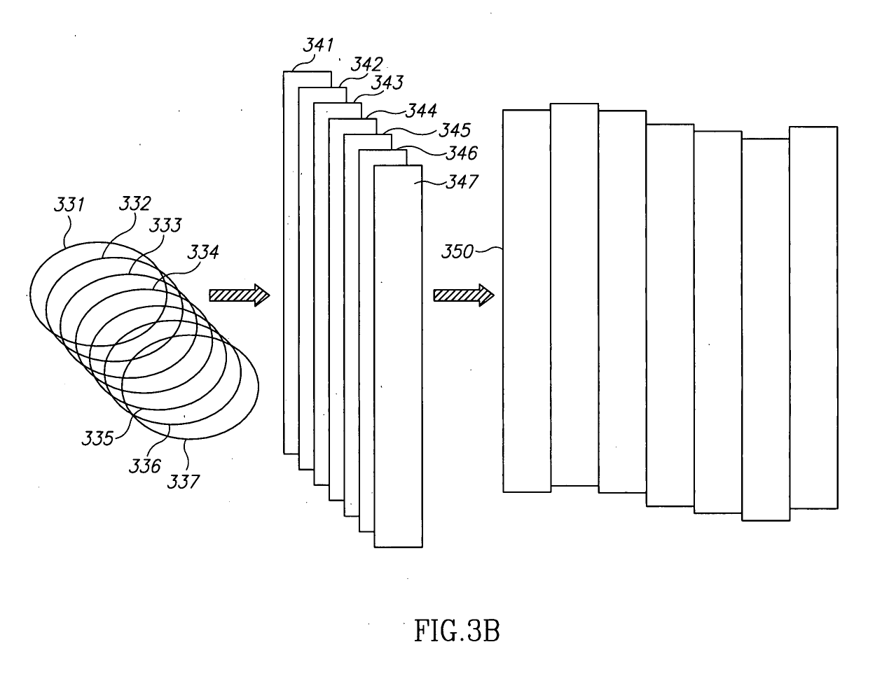

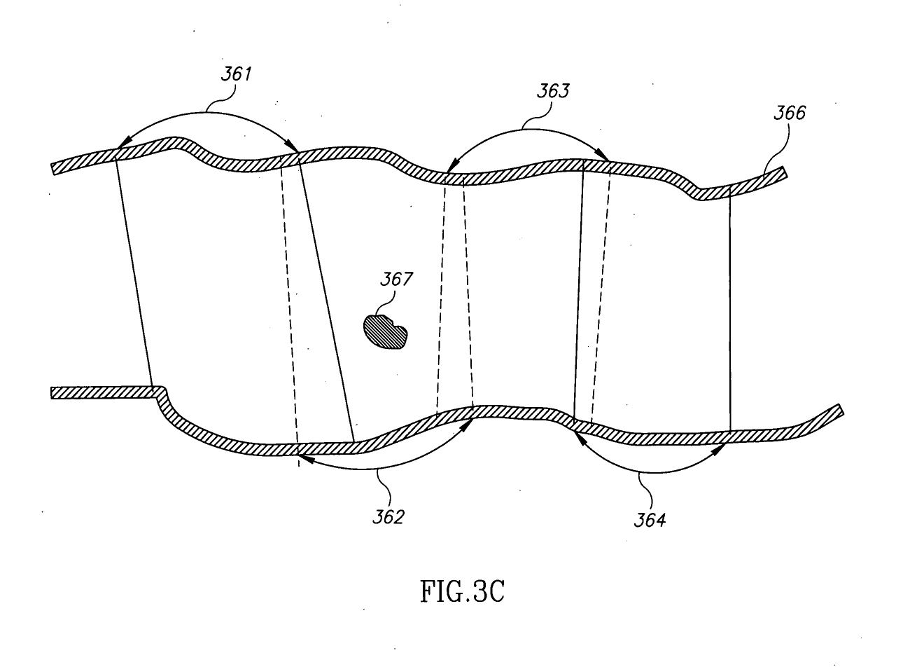

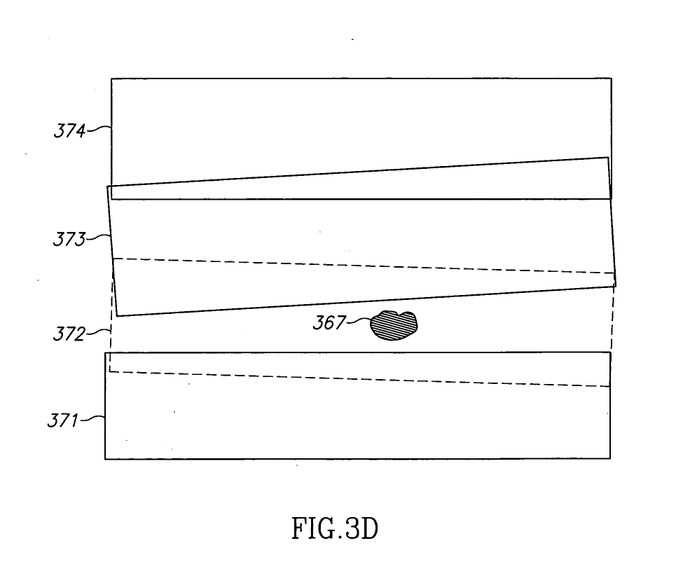

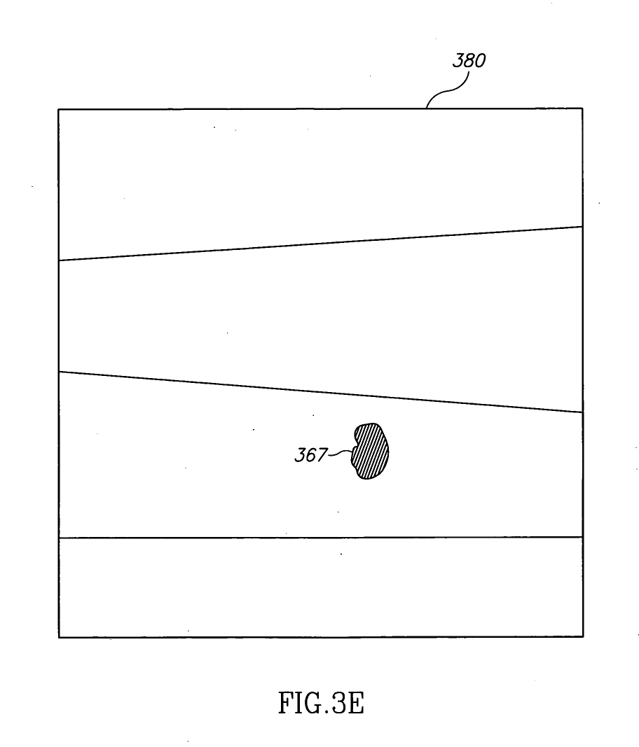

FIG. 3A schematically illustrates the combination of a plurality of images of slices 311, 312, 313, 314, 315, 316, 317 and 318, into a combined image 320 in accordance with embodiments of the invention as described above.

FIG. 3B schematically illustrates the conversion of a plurality of circular slice or ring shaped images 331, 332, 333, 334, 335, 336 and 337 into a plurality of rectangular images of slices 341, 342, 343, 344, 345, 346 and 347 in accordance with embodiments of the invention as described above. FIG. 3B further schematically illustrates the combination of a plurality of rectangular images of slices 341, 342, 343, 344, 345, 346 and 347 into a combined image 350 in accordance with embodiments of the invention as described above.

In some embodiments, imager 46 and/or device 40 may be controlled and/or programmed, for example, to allow capturing a continuous “chain of images” representing a body lumen. In one embodiment, consecutive images may partially cover one area of the body lumen, for example, such that images may partially overlap. In some embodiments, for example, image capture rate may be pre-defined and/or controlled in real-time, to allow imager 46 and/or device 40 to capture a continuous “chain of images”. In one embodiment, a suitable image correlation technique may be used, for example, to detect and/or process overlapping areas among images, or to combine a plurality of images into a combined image.

FIG. 3C schematically illustrates a “chain of images” of body lumen 366 in accordance with some embodiments of the invention. In one embodiment, images 361, 362, 363 and 364 may be captured by imager 46. As illustrated schematically in FIG. 3C, the images may partially overlap. For example, image 362 may include a portion of body lumen 366 captured in image 361 and/or a portion of body lumen 366 captured by image 363. Image 362 may additionally include an image of item 367, for example, a body organ, a material, a blood, a pathology, etc.

FIG. 3D schematically illustrates an alignment of images in accordance with some embodiments of the invention. For example, in one embodiment, the four images 361, 362, 363 and 364 of FIG. 3C may be processed, correlated and/or aligned, to produce four aligned images 371, 372, 373 and 374, respectively. It is, noted that aligned image 372 may include, for example, the image of item 367.

FIG. 3E schematically illustrates a combination of images in accordance with some embodiments of the invention. For example, in one embodiment, the four images 361, 362, 363 and 364 of FIG. 3C, and/or the four images 371, 372, 373 and 374 of FIG. 3D, may be processed, correlated and/or aligned, to produce a combined image 380. It is noted that combined image 380 may include, for example, the image of item 367.

It is noted that FIGS. 3A to 3E include exemplary illustrations only, and that the present invention is not limited in this regard. In alternate embodiments, other suitable methods for capturing, converting, combining, matching, aligning, processing, correlating and/or displaying images may be used; for example, a relatively continuous “spiral” image or series of images may be captured and/or displayed, a discontinuous series of “slices” may be captured and/or displayed, etc. Images need not be combined or processed before display.

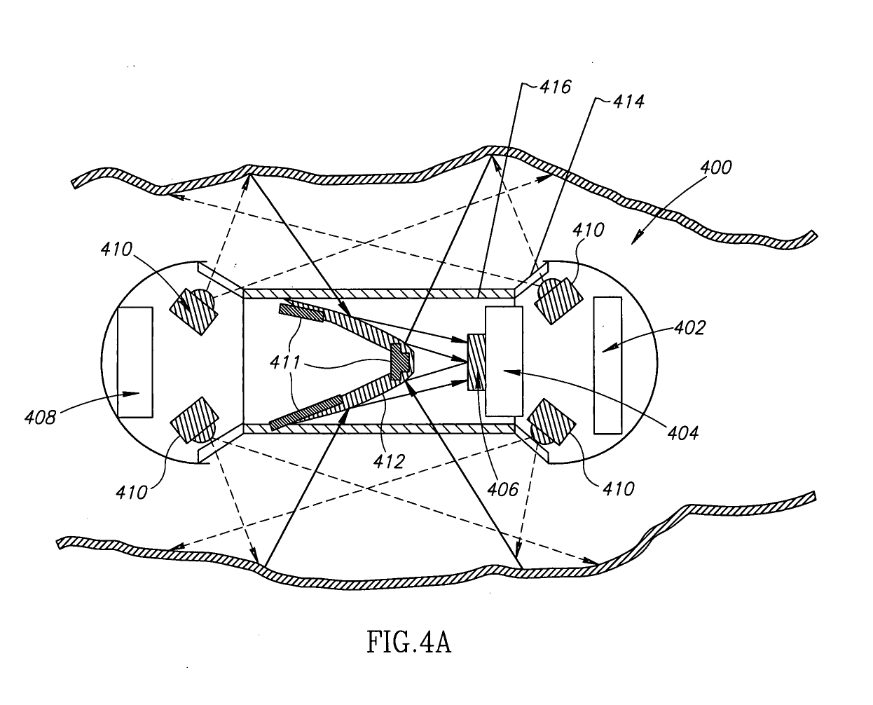

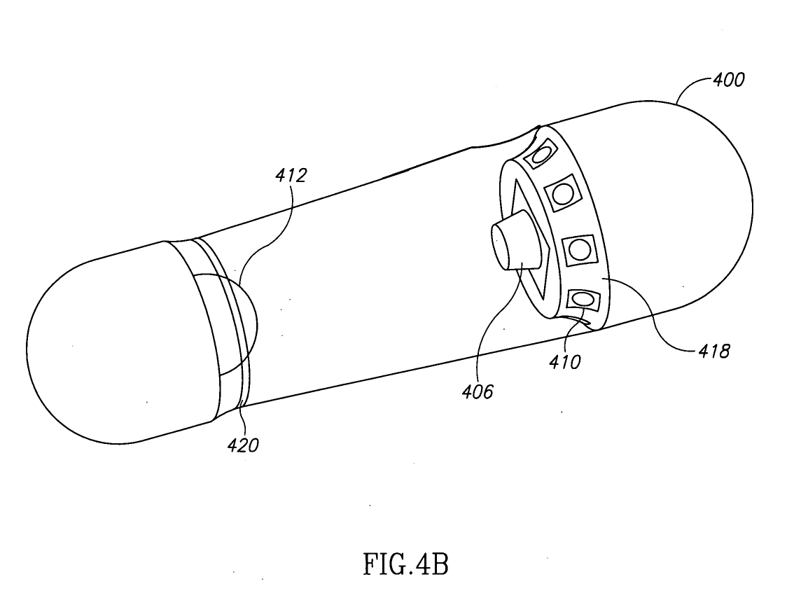

Reference is made to FIG. 4A, a schematic diagram of an in-vivo imaging device with a narrowed section in accordance with an embodiment of the invention. Device 400 may include elements and/or may operate for example as described in FIG. 2 of this application. For example, device 400 may include a transmitter and an antenna 402, a processor 404, an image sensor 406, a power supply 408, one or more illuminators 410 and a reflective element such as for example a mirror 412 or a curved mirror. Mirror 412 may be held in place by for example anchors 411. Portions of for example an outer shell of device 400, such as for example a narrowed portion of device 400, may be transparent to the light emitted by illuminators 410. For example, section 414 of device 400 may be a transparent portion of an outer shell of device 400 in front of illuminator 410. Section 414 may allow light (indicated by dashed lines) emitted by illuminator 410 to exit device 400 and reach an endo-luminal area. Section 414 may be angled to form part of a tapered section between one or more wider ends of device 400 and a narrower transparent ring 416. In some embodiments the transparent ring 416 may be in the shape of a partial ring or a window or other shape. Transparent ring 416 may for example be transparent to the light emitted by illuminators 410 that is reflected back off of for example an endo-luminal wall (as indicated by solid lines) to device 400. According to one embodiment device 400 maintains a capsule like shape, which may be advantageous for movement in-vivo however, the transparent ring 416 may be configured such that an appropriate field of illumination of the body lumen walls may be achieved with a reduced risk of stray light or backscatter from illumination sources 410 onto the image sensor 406.

Device 400 may, in some embodiments, capture a panoramic (such as, for example, 360 degrees) or partially panoramic view of an in-vivo area. According to one embodiment, illuminators 410 may be substantially contiguous with transparent section 414 and transparent ring 416 such that no or few light rays emitted from the illumination sources 410 are backscattered onto image sensor 406, but rather they are incident on the body lumen walls and can be reflected onto image sensor 406. According to one embodiment, illuminators 410 are positioned behind section 414 of transparent ring 416, which may be typically beveled or at an angle to transparent ring 416, so as to enable an unobstructed field of illumination on the body wall being imaged, but so as not to obstruct light rays remitted from the body lumen wall onto the imager.

In some embodiments, an area of an imaging device 400 may be concave, tapered, narrowed or ‘pinched’ so that the device may have a shape resembling a peanut. Such concave area may for example include transparent ring 416, segment or viewing window through which light may enter and be reflected off of mirror 412 onto an image sensor 406. In some embodiments, mirror 412 may be in a parabolic shape, such that for example light rays striking mirror 412 from various directions will be reflected towards image sensor 406. In some embodiments, the peanut shape may minimize the backscatter light that reaches the image sensor 406 directly from illuminators 410 rather than after being reflected off of endo-luminal wall.

Reference is made to FIG. 4B, a schematic diagram of a ring of light emitting diodes (LEDs) or illuminators 410 that may be on a ring that is slanted outward in relation to the plain of an image sensor 406 in accordance with an embodiment of the invention. Illuminators 410 may be situated for example on an outward facing ring 418 such that illuminators 410 face outward and away from image sensor 406. Placement of illuminators 410 on ring 418 as it is slanted outward and away from image sensor 406 may avoid backscatter of light directly from illuminators onto image sensor 406. In another embodiment, a second reflective element 420 may be situated behind mirror 412 so as to reflect onto an endo-luminal wall light that may be emitted directly from illuminators 410 and that might otherwise not reach endo-luminal wall.

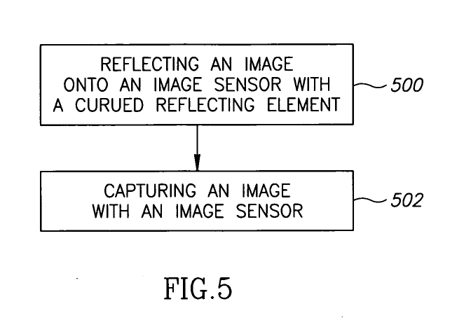

FIG. 5 is a flow chart diagram of a method of capturing an image using a curved reflective element in accordance with embodiments of the invention. In one embodiment, device 200 may traverse body lumen 271. As is indicated in block 500, an image of an in-vivo area may be reflected onto an imager 46 or image sensor by way of a curved reflective element 260. In block 502 the reflected image may be captured by the imager 46. Imager 46 may capture images of portions of body lumen 271, for example, of slice 272.

The images may be processed and/or converted and/or combined, for example using processing unit 47 or, typically after transmission, using an external processor such as processor 14. In some embodiments, the images may be transmitted using transmitter 41 and antenna 48. Other transmission methods may be used.

The image may be received by receiver 12 and may be transferred to data processor 14. The image may be displayed and/or stored in storage unit 19.

Other operations or series of operations may be used. The above operations may be repeated as desired, for example, until a pre-defined period of time elapses, and/or until a pre-defined number of images are taken, and/or until the imaging device exits the patient's body, until a user instructs the system to discontinue repeating the above operations, and/or until another pre-defined condition and/or criteria are met.

Additionally or alternatively, if desired, a captured image or a plurality of captured images may be converted, for example, from a circular and/or ring shape into a rectangular shape. Additionally or alternatively, if desired, a plurality of captured images and/or converted images may be combined into one or more combined images of, for example, body lumen 271 (FIG. 2). The captured images, the converted images and/or the combined images may be displayed, for example, using monitor 18.

Additionally or alternatively other operations may be performed with the captured images, the converted images and/or the combined images, for example, to store such images using various types of storage devices, to print such images using a printer, to perform operations of image manipulation and/or enhancement, to perform operations of video manipulation and/or enhancement, or the like.

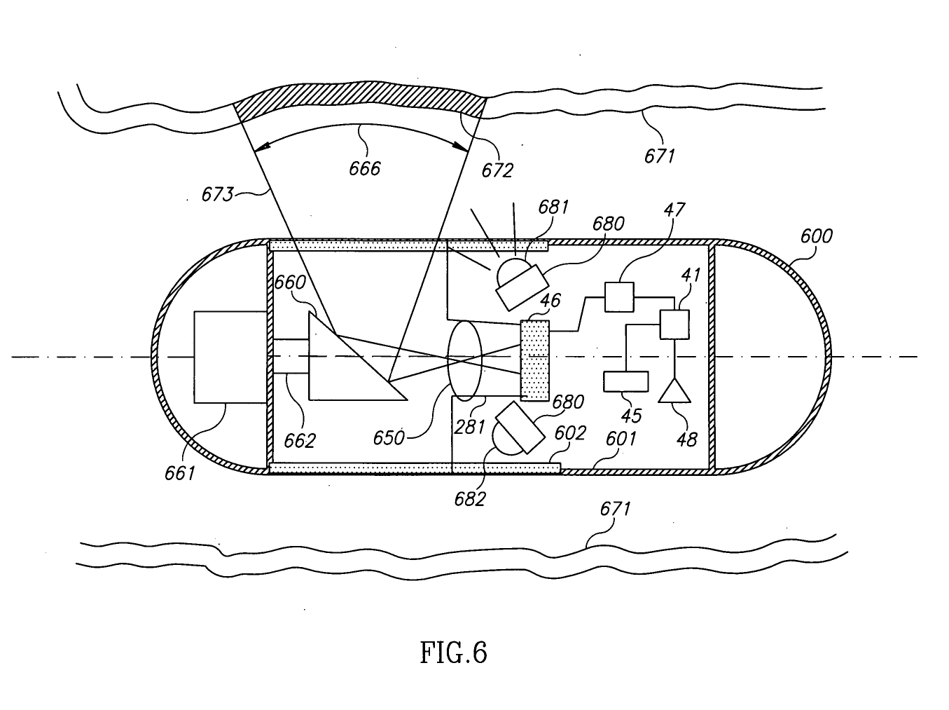

FIG. 6 is a schematic illustration of an in-vivo imaging device 600 in accordance with embodiments of the invention. Device 600 may be an implementation or variation of device 40, and may be used, for example, in conjunction with the system of FIG. 1. For example, device 600 may be used in conjunction with receiver 12 and/or data processor 14. In one embodiment of the invention, device 600 may be implemented as, for example, a swallowable capsule and may include, for example, an imager 46, a processing unit 47, a transmitter 41, an antenna 48, a power source 45, a lens assembly 650, a mirror or reflective device 660, one or more illumination sources 680, and a holder 281. The reflective device 660 may further include a motor 661 and a shaft 662.

In one embodiment of the invention, device 600 may be a swallowable capsule. Device 600 may be partially or entirely transparent. For example, device 600 may include one or more areas and/or portions, such as a transparent shell or portion 602, which are transparent and which allow components inside device 600 to have an un-obstructed field-of-view of the environment external to device 600. In alternate embodiments, transparent areas and/or portion may have different shapes.

Lens assembly 650 may include, for example, one or more lenses or optical systems which allow images reflected by mirror 660 to be focused onto imager 46. Additionally or alternatively, lens assembly 650 may include a combination of lenses able to zoom in and/or zoom out on an image or on several parts of an image reflected by mirror 660. Lens assembly 650 may include one or more optical elements, for example, one or more lenses and/or optical filters, to allow or to aid focusing reflected light onto imager 46 and/or performing other light processing operations.

Mirror 660 may include, for example, a glass and/or metal mirror or any other suitable reflective surface. Mirror 660 may be placed, positioned and/or aligned to allow a slice 672 or other portion of a body lumen 671 to be reflected by mirror 660, through lens assembly 650, onto imager 46. For example, mirror 660 may be situated at a 45 degree angle to the plane of imager 46 or to the plane of transparent shell 602. It is noted that other angles may be used to achieve specific functionalities and/or to allow imager 46 a broader or narrower field-of-view. Further, in some embodiments, other arrangements and/or series of optical elements may be used, and functionalities, such as reflecting and/or focusing, may be combined in certain units.

Illumination sources 680 may include one or more illumination sources or light sources to illuminate body lumen 671 and/or a slice 672 of body lumen 671. In one embodiment, illumination sources 680 may include one or more Light-Emitting Diodes (LEDs), for example, one or more white LEDs. Such LEDs may be placed, aligned and/or positioned to allow a desired illumination of body lumen 671, for example, using a ring-shaped arrangement of LEDs able to illuminate body lumen 671 through transparent shell 602. In some embodiments of the present invention, one or more illumination sources 680 may be positioned in a slanted orientation.

Motor 661 may include an electro-mechanical motor able to rotate shaft 662 which may be attached to motor 661, and mirror or reflective device 660 which may be attached to shaft 662. The rotation rate of motor 661 may be constant or variable. The rotation rate of motor 661 may be, for example, 250 rotations per minute; other constant and/or variable rotation rates may be used. It is noted that when motor 661 rotates shaft 662 and mirror or reflective device 660, the field-of-view of imager 46 may change respectively, such that the instantaneous field-of-view 666 of imager 46 may include a part of slice 672 of body lumen 671. Additionally or alternatively, in one rotation of mirror 660, the field-of-view of imager 46 may include substantially an entire ring-shaped slice 672 of body lumen 671. Motor 661 may be controlled by, for example, transmitter 41; in alternate embodiments another unit such as a separate controller may provide such control.

In one embodiment, as device 600 traverses body lumen 671, device 600 may capture images of a slice of body lumen 671, such as slice 672. Illumination sources 680 may illuminate slice 672 of body lumen 671 when slice 672 is in the instantaneously field-of-view of imager 46. The light from illuminated slice 672 may be reflected using mirror or reflected surface 660, focused and/or transferred using lens assembly 650, and received by imager 46 which may thereby capture an image of slice 672. In alternate embodiments, other suitable methods for capturing images and/or displaying images may be used; for example, a relatively continuous “spiral” image or series of images may be captured, a discontinuous series of “slices” may be captured, etc.

In some embodiments, sets of illumination sources 680 may be turned on and/or turned off substantially simultaneously, such that substantially all illumination sources 680 are either turned on or turned off at a given point in time.

In other embodiments, some of illumination sources 680 are turned on and some of illumination sources 680 are turned off at a given point in time. For example, in one embodiment, illumination sources 680 may be configured to be in synchronization with rotation of motor 661 and/or mirror or reflective surface 660, such that the field of illumination created by illumination sources 680 creates sufficient light to illuminate the instantaneous field-of-view of imager 46.

In some embodiments, illumination sources 680 may include a ring of light sources such as LEDs, for example, LEDs 681 and 682; some LEDs, for example, LED 681, may be turned on when other LEDs, for example, LED 682, are turned off, or vice versa. In one embodiment, illumination sources 680 may include a ring of LEDs, such that each LED may be synchronously on when the instantaneous field-of-view of imager 46 covers and/or overlaps the field of illumination of that LED. Of course, illumination sources other than LEDs may be used in accordance with embodiments of the invention.

In some embodiments, an optional optical system (not shown) may be used in conjunction with illumination source 680, for example, to create a desired illumination, for example, homogenous illumination, of an imaged body lumen. In one embodiment, the optical system may include, for example, one or more mirrors and/or curved mirrors and/or lenses and/or reflective elements, and/or filters shaped and/or positioned and/or aligned to create a desired, e.g., homogenous, illumination. For example, in one embodiment, the optical system may include a curved mirror, similar to reflective element 260 of FIG. 2.

In one embodiment, since device 600 may include transparent areas, such as transparent shell 602, the captured image may include a reflected image of a ring-shaped slice 672 of body lumen 271. It is noted that lens assembly 650 may be configured, placed and/or aligned to filter and/or focus light from body lumen 671, such that only light from a desired portion of body lumen 671, for example, a ring-shaped slice 672, falls on imager 46. Using device 600 may allow capturing a panoramic image of slice 672 of body lumen 671. Such panoramic image may include a substantially complete 360 degrees image of slice 672. Alternatively, if desired, such image may include a non-complete image of slice 672, for example, a 270 degrees image, a 180 degrees image, or other wide angle or partially panoramic images of a body lumen.

In one embodiment, the panoramic image of slice 672 may be ring-shaped. Such an image may be converted into a rectangular image of slice 672 or into other shapes as is described elsewhere in this application.

Images of slices of body lumen 671, such as slice 672, may be placed, aligned and/or combined together, for example, side by side, to create a combined image or several combined images from a plurality of images of slices. The combination of images of slices may be performed, for example, by processing unit 47 and/or data processor 14. Additionally or alternatively, the combination of images of slices may be performed before and/or after transmission of the image by transmitter 41 to receiver 12.

In one embodiment, imager 46 may capture one or more images of body lumen 671 per rotation of motor 661. Other capture rates, constant or variable, may be used. In one embodiment, imager 46 may continuously remain active and/or receive light to take one image per rotation of motor 661.

In some embodiments, device 600 may further include one or more additional sets of imager and lens, to take images of other areas of body lumen 671 in addition to the images taken using imager 46. For example, device 600 may include an additional imager or several additional imagers (not shown), which may be positioned to obtain a field-of-view different (e.g., broader) from the field-of-view of imager 46. In some embodiments, imager 46 may include one or more imagers positioned to cover a broader field-of-view, for example, three or four imagers in a circular configuration aimed towards body lumen 671.

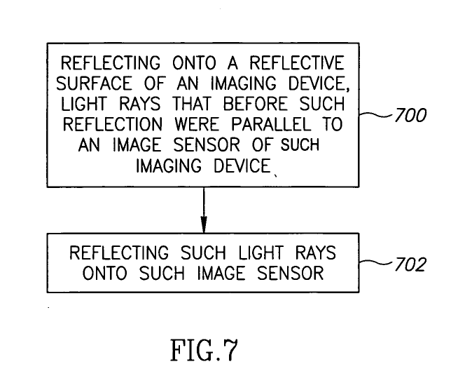

Reference is made to FIG. 7, a flow chart of a method of reflecting light rays onto an imager 46 in accordance with an embodiment of the invention. In block 700, light rays 673 may be reflected onto a mirror or reflective device 660 of device 600. Some of such light rays 673 before such reflection may have been parallel or substantially parallel to a plane of an imager 46 of imaging device 600 upon which light detection sensors may be located. In block 702, the lights rays 673 may be reflected off of a mirror or reflective surface 660 and onto imager 46. In an embodiment of the invention, mirror or reflective surface 660 may be situated at an angle, such as for example a 45 degree angle to the imager 46. Other angles may be used. In some embodiments, mirror or reflective surface 660 may be rotated by for example a motor 661, and there may be reflected onto imager 46 a panoramic or partially panoramic image of an in-vivo are surrounding the device 600. In some embodiments illumination sources 680 may direct light through a transparent portion of the imaging device onto an in-vivo area.

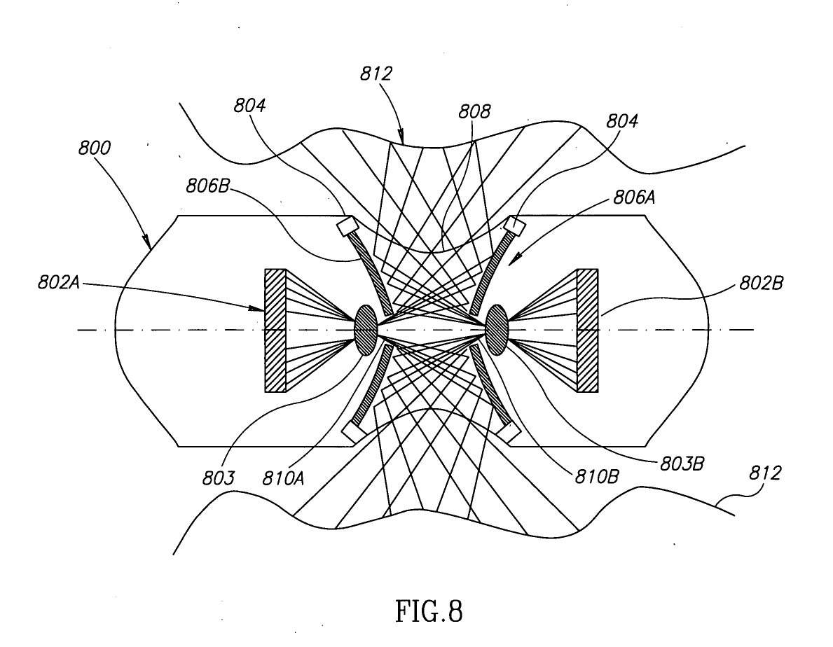

Reference is made to FIG. 8, a depiction of a panoramic capsule in accordance with an embodiment of the invention. Device 800 may include one or more image sensors 802, one or more lenses 803, and one or more illumination sources 804. In some embodiments, one or more of mirrors 806, such as, for example, curved mirrors or mirrors shaped in a parabolic and/or conic form may be situated facing each other between a tapered section or concave ring 808 of the outer shell of device 800. One or more of lenses 803 may be situated behind an opening or space in mirrors 806 such that light reflected off of a mirror 806A passes through space 810A towards lens 802A, and light reflected off mirror 806B may pass through space 810B towards lens 803B. Device 800 may in some embodiments be suitable to capture a three dimensional and panoramic view of endo-luminal walls 812.

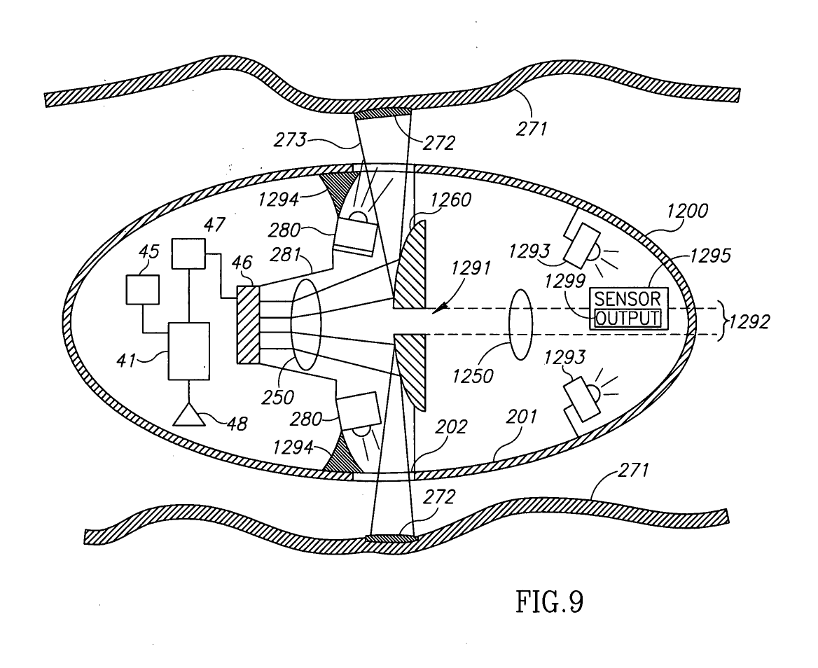

FIG. 9 schematically illustrates an in-vivo imaging device 1200 able to acquire images from multiple sources or from multiple fields-of-view, in accordance with some embodiments of the invention. Device 1200 may be an implementation or variation of device 40, and may be used, for example, in conjunction with the system of FIG. 1 or certain components of FIG. 1. For example, device 1200 may be used in conjunction with receiver 12 and/or data processor 14. In one embodiment, for example, device 1200 may be similar to device 200 of FIG. 2, and may include, for example, imager 46, processing unit 47, transmitter 41, antenna 48, power source 45, lens assembly 250, a reflective element 1260, an illumination source (or plurality of sources) 280, and a holder 281. The processing capability of processing unit 47 may be combined with other units, such as transmitter 41 or a separate controller. Device 1200 need not be similar to devices 40 or 200.

In some embodiments, the reflective element 1260 may include, for example, a curved mirror having an aperture 1291, e.g., a hole, an orifice, a space, a cavity, a window, a transparent portion, a slit, or the like. In some embodiments, reflective element 1260 may include, for example, a metallic element, a reflective plastic element, a reflective coated plastic element, or a glass element. Reflective element 1260 may be shaped and/or contoured such that it may allow light reflected from slice 272 of body lumen 271 to be reflected by reflective element 1260, through lens assembly 250, onto imager 46. For example, reflective element 1260 may be oval, spherical, radial, circular, ellipse-shaped, faceted, conical, etc. Other shapes may be used. It is noted that in some embodiments, reflective element 1260 may have a shape, size and/or dimensions to allow a desired reflection of light and/or to allow a desired range and/or field-of-view. In one embodiment, reflective element 1260 may be manufactured using suitable optical design software and/or ray-tracing software, for example, using “ZEMAX Optical Design Program” software. Other suitable shapes may be used.

In some embodiments, aperture 1291 may be located substantially central to reflective element 1260, for example, in a substantially central “dead” area where rays reflected from a slice 272 may not fall. Aperture 1291 may be circular, oval, rectangular, square-shaped, or may have other suitable shapes. In some embodiments, two or more apertures 1291 may be used. Other positions and/or shapes for the one or more apertures 1291 may be used.

Aperture 1291 may allow passage of light rays, e.g., reflected from an object or body lumen located in frontal viewing window and/or area 1292. In one embodiment, such object or body lumen may be illuminated, for example, using one or more illumination units 1293, and/or using other illumination devices, e.g., illumination ring 418 of FIG. 4. In other embodiments, a reflective surface 1294 may be used to reflect light from illumination source 280 toward a viewing area to be viewed from frontal viewing window 1292. Other configurations may be used for illumination in the frontal viewing window and/or area 1292. It is noted that frontal viewing window and/or area 1292 is used herein as a relative term, and may be any viewing window and/or area substantially perpendicular to the panoramic viewing window and/or area.

In some embodiments, a first illumination unit (e.g., illumination unit 280) may be located at a first location of the in-vivo device 1200, may be oriented or directed at a first orientation or direction (e.g., directed towards a body lumen, or substantially perpendicular to the imager 46), and may illuminate a first field of view, e.g., a field of view of a first portion of a body lumen (e.g., slice 272); whereas a second illumination unit (e.g., illumination unit 1293) may be located at a second location of the in-vivo device 1200, may be oriented or directed at a second orientation or direction (e.g., directed towards another portion of the body lumen, or substantially frontal to the imager 46), and may illuminate a second field of view, e.g., a field of view of a second portion of a body lumen (e.g., slice 272) and/or a field of view including the in-vivo sensor 1295 or a visual output 1299 thereof.

In some embodiments, the light rays reflected from the object or body lumen located in frontal field-of-view 1292 may optionally pass through a lens assembly or optical system 1250, for example, before they pass through the aperture 1291, e.g.; to focus the light rays. In other embodiments, lens or lens system 1250 may be positioned anywhere between imager 46 and frontal viewing window 1292, for example, the lens system 1250 may be fitted onto aperture 129. The lens assembly or optical system 1250 may be entirely or partially within the frontal field-of-view 1292, or may be entirely or partially outside the frontal field of view 1292.

Upon passage through the aperture 1291, the light ray may pass through the lens assembly 250 and may be captured by the imager 46.

In some embodiments, the imager 46 may acquire images having multiple portions. For example, an image acquired by the imager 46 may include a first (e.g., external, ring-shaped, or other shaped) portion showing an image captured from light reflected by the reflective element 1260, and a second (e.g., circular, internal) portion showing an image captured from light passing through the aperture 1291.

In some embodiments, instead of or in addition to imaging a body lumen through aperture 1291, the imager 46 may capture visual information from, for example a sensor 1295 of the in-vivo device 1200. For example, sensor 1295 may include a pH sensor, a temperature sensor, a liquid crystal temperature sensor, an electrical impedance sensor, a pressure information, a biological sensor (e.g., able to sense or analyze a collected sample), or other suitable sensor. Sensor 1295 may include, for example, a fixed or non-mechanical substance that reacts in a visual manner to its environment, such as registering or indicating pH, temperature, pressure, one or more substances, etc. Sensor 1295 may be able to produce a visual output or visual indication in response to the data sensed by sensor 1295, for example, change in color, change in light intensity, change in shape, etc. In some embodiments, for example, sensor 1295 may produce visual output, for example, through an optional visual output sub-unit 1299, which may include, for example, a part or portion of sensor 1295. For example, the visual output sub-unit 1299 of sensor 1295 may include, for example, a liquid crystal sensor able to display or output one or more values or colors, e.g., sensor 1295 may display a sensed value, or may present a color (e.g., red, orange, yellow, or the like) in response to sensing. In one embodiment, imager 46 may acquire images (e.g., through aperture 1291) of sensor 1295, and/or of visual output sub-unit 1299, and/or of a portion or part of sensor 1295 which otherwise produces visual output. Other methods of producing and acquiring sensor output and/or illumination may be implemented.

In some embodiments of the present invention, one or more sampling chambers and/or one or more sensors that may perform biological sensing of the one or more sampling chambers may be imaged through lens system 1250 by imager 46. In one embodiment, a reaction occurring in a sampling chamber may result in a color or other visual indication. For example, antibodies may be directed against, for example, different antigenic determinants or other determinants and the binding of the antibody and, for example, antigenic determinants may directly or indirectly result in a color and/or other visual indication that may be imaged through aperture 1291 and/or in the vicinity of aperture 1291. Other biological sensing may be performed and/or imaged, for example, in other manners. In one embodiment of the present invention, a sampling chamber may be positioned in, in front of, or in proximity to, aperture 1291 such that it may be imaged by imager 46. In other embodiments, a sampling chamber positioned in or near aperture 1291 may be sensed by other sensing means, for example, by a magnetic field sensor. According to some embodiments, lens system 1250 may provide microscopic imaging capability and, for example, one or more sampling chambers may be directed substantially near lens system 1250 so that a microscopic image may be captured of one or more sampled medium. In another embodiment, sensor 1295 may be a “lab on chip device” that may be imaged by imager 46 through, for example, lens system 1250. Aperture 1291 and lens system 1250 may be implemented to image other suitable sources of information.

In some embodiments, for example, aperture 1291 may allow passage of light rays, e.g., reflected from or passing through or produced by the sensor 1295. In one embodiment, the sensor 1295 may be illuminated, for example, using one or more illumination units 1293, and/or using other illumination devices, e.g., illumination ring 418 of FIG. 4. In some embodiments of the present invention, fiber optics may be used to direct light from, for example, illumination source 280 to the sensor 1295 area to, for example, illuminate the sensor 1295 output. In other embodiments of the present invention, an optional reflective surface 1294, for example a reflective ring, may direct light toward the direction of viewing window 1292. Other methods of illuminating a secondary and/or alternate viewing direction may be implemented.

In some embodiments, the light rays reflected from the sensor 1295 may optionally pass through lens assembly or optical system 1250 before they pass through the aperture 1291, e.g., to focus the light rays.

In some embodiments, an image acquired by the imager 46 may include a first (e.g., external, ring-shaped or other shaped) portion showing an image captured from light reflected by the reflective element 1260, and a second (e.g., internal or central) portion showing an image captured from light reflected by the sensor 1295.

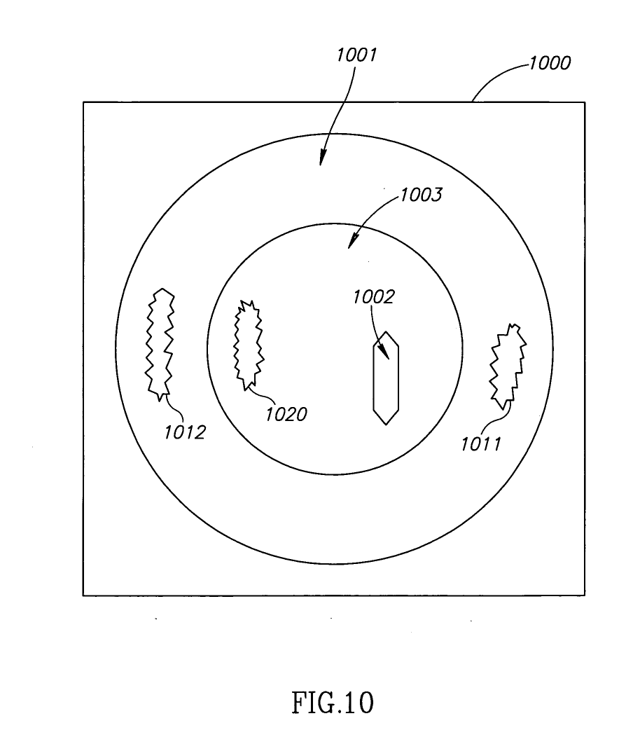

Reference is now made to FIG. 10, which schematically illustrates an exemplary image 1000 which may be captured by the in-vivo imaging device 1200 of FIG. 9 from a plurality of sources or from a plurality of fields-of-view. Image 1000 may include, for example, a first (e.g., external, ring-shaped or other shaped) portion 1001 showing an area or image-portion captured from light reflected by the reflective element 1260; a second (e.g., internal or central) portion 1002 showing an area or image-portion captured from light reflected by the sensor 1295 or by the visual output sub-unit 1299 of sensor 1295; and a third (e.g., internal or central) portion 1003 showing an area of image-portion captured from light reflected from an object or lumen located at the frontal field of view 1292.

In some embodiments, image 1000 may include multiple image-portions, for example, a first image-portion (e.g., portion 1001) corresponding to a first field-of-view (e.g., panoramic field-of-view) or a first source or object (e.g., a first portion or slice of a body lumen), and a second image-portion (e.g., portion 1003) corresponding to a second field-of-view (e.g., frontal field-of-view) or a second source or object (e.g., a second portion or slice of a body lumen, or a visual output of an in-vivo sensor). In some embodiments, an image-portion may include, or may correspond to, for example, a part of an image, a field-of-view, an area, an imaged area, an area of interest. For example, image 1000 may include multiple image-portions, such that the size of a portion may be smaller than the size of image 1000. Although image 1000 is shown, for demonstrative purposes, to include three image portions 1001-1003, other number of image portions may be included in image 1000, e.g., corresponding to other numbers, respectively, of fields-of-view, areas-of-interest, imaged areas, imaged objects, or the like. In some embodiments, optionally, multiple image-portions may correspond to multiple objects or may include multiple objects, for example, multiple portions or slices of a body lumen, multiple areas of a body lumen, visual output(s) of one or more in-vivo sensors, multiple objects located in multiple fields of view, respectively, or the like.

In the example shown in FIG. 10, portion 1003 may include an imaged object 1020 (e.g., an object or a portion of body lumen) which may be located in the frontal field-of-view and viewed from frontal window 1292 of FIG. 9; and portion 1001 may include objects 1011 and 1012 (e.g., objects or portions of body lumen) of slices 272 of FIG. 9. Other suitable objects or portions may be imaged, and other suitable fields-of-view may be used; fields of view produced by embodiments of the invention may have other arrangements.

In one embodiment, image 1000 may include three image portions 1001, 1002 and 1003; in other embodiments, image 1000 may include other number of image portions. In one embodiment, image portion 1001 may be, for example, ring-shaped and may surround image portions 1002 and 1003; in other embodiments, other suitable shapes and arrangements may be used.

Although portions of the discussion herein may relate, for example, to a first field of view which may be substantially perpendicular to the imager and a second field of view which may be substantially frontal to the imager, other suitable fields of view may be used and/or combined (e.g., within an in-vivo image) in accordance with embodiments of the invention, for example, a field of view at an angel of approximately 1.5 degrees relative to the imager, a field of view at an angel of approximately 30 degrees relative to the imager, a field of view at an angel of approximately 45 degrees relative to the imager, a field of view at an angel of approximately 60 degrees relative to the imager, a field of view at an angel of approximately 75 degrees relative to the imager, a field of view at an angel of approximately 90 degrees relative to the imager, a field of view at an angel of approximately 105 degrees relative to the imager, a field of view at an angel of approximately 120 degrees relative to the imager, a field of view at an angel of approximately 135 degrees relative to the imager, a field of view at an angel of approximately 145 degrees relative to the imager, a field of view at an angel of approximately 160 degrees relative to the imager, or the like. Other suitable angles or directions may be used.

While some features are described in the context of particular embodiments, the invention includes embodiments where features of one embodiment described herein may be applied to or incorporated in another embodiment. Embodiments of the present invention may include features, components, or operations from different specific embodiments presented herein.

While certain features of the invention have been illustrated and described herein, many modifications, substitutions, changes, and equivalents may occur to those of ordinary skill in the art. It is, therefore, to be understood that the appended claims are intended to cover all such modifications and changes as fall within the true spirit of the invention.

What is claimed is:

1. An in-vivo imaging device comprising:
an imager able to acquire an in-vivo image including at least a first image-portion and a second image-portion, the first image-portion corresponding to a first field of view of the in-vivo imaging device, and the second image-portion corresponding to a second field of view of the in-vivo imaging device.

2. The in-vivo imaging device of claim 1, wherein the first field of view is a panoramic field of view.

3. The in-vivo imaging device of claim 1, wherein the first field of view is substantially perpendicular to the imager.

4. The in-vivo imaging device of claim 1, wherein the second field of view is a frontal field of view.

5. The in-vivo imaging device of claim 1, wherein the first image-portion is substantially ring shaped and the second image-portion is substantially circular.

6. The in-vivo imaging device of claim 1, wherein the first image-portion substantially surrounds the second image-portion.

7. The in-vivo imaging device of claim 1, further comprising:
a curved reflective element to reflect light onto the imager from the first field of view.

8. The in-vivo imaging device of claim 7, wherein the curved reflective element comprises an aperture to allow passage of light from the second field of view onto the imager.

9. The in-vivo imaging device of claim 8, wherein the aperture is substantially central to the curved reflective element.

10. The in-vivo imaging device of claim 1, wherein the first field of view includes a first portion of a body lumen substantially perpendicular to the imager, and wherein the second field of view includes a second portion of the body lumen substantially frontal to the imager.

11. The in-vivo imaging device of claim 1, wherein the first field of view includes a first object and the second field of view includes a second object.

12. The in-vivo imaging device of claim 11, wherein the first object is external to the in-vivo imaging device, and wherein at least a portion of the second object is internal to the in-vivo imaging device.

13. The in-vivo imaging device of claim 1, further comprising an in-vivo sensor able to generate a visual output.

14. The in-vivo imaging device of claim 13, wherein the first field of view includes a portion of a body lumen, and wherein the second field of view includes at least a portion of the visual output of the in-vivo sensor.

15. The in-vivo imaging device of claim 1, further comprising an illumination source to illuminate the first and second fields of view.

16. The in-vivo imaging device of claim 15, wherein the illumination source comprises:
a first illumination unit at a first orientation to illuminate the first field of view; and
a second illumination unit at a second orientation to illuminate the second field of view.

17. The in-vivo imaging device of claim 1, wherein the in-vivo imaging device is autonomous.

18. The in-vivo imaging device of claim 1, comprising a swallowable capsule.

19. An in-vivo imaging system comprising:
an in-vivo imaging device comprising:
an imager able to acquire an in-vivo image including at least a first image-portion and
a second image-portion, the first image-portion corresponding to a first field of view of the in-vivo imaging device, and the second image-portion corresponding to a second field of view of the in-vivo imaging device; and
a transmitter to transmit the in-vivo image data.

20. The in-vivo system device of claim 19, wherein the first field of view is a panoramic field of view, and wherein the second field of view is a frontal field of view.

21. The in-vivo imaging system of claim 19, further comprising:
a receiver to receive the in-vivo image data; and
a monitor to display the in-vivo image data.

22. The in-vivo imaging system of claim 19, wherein the in-vivo imaging device is autonomous.

23. The in-vivo imaging system of claim 19, wherein the in-vivo imaging device comprises a swallowable capsule.
